(19) United States
(12) Patent Application Publication
(10) Pub. No.: US 2009/0233380 A1
(43) Pub. Date: Sep. 17, 2009

(54) Methods and Compositions of Conjugating Gold to Biological Molecules

(75) Inventors: Michael Belmares, Sunnyvale, CA (US); Carol Tan, Sunnyvale, CA (US); Lilyn Liu, Sunnyvale, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Arbor Vita Corporation, Sunnyvale, CA (US)

(21) Appl. No.: 12/359,270

(22) Filed: Jan. 23, 2009

Related U.S. Application Data
(60) Provisional application No. 61/023,619, filed on Jan. 25, 2008.

(51) Int. Cl. G01N 33/553; C07K 1/13; G01N 33/20; C07K 16/00

(52) U.S. Cl. 436/525; 530/345; 436/76; 530/391.3

(57) ABSTRACT

The present application describes methods for conjugating gold to biological molecules and conjugates resulting from the same. The method provides superior gold conjugated biomolecules with higher sensitivity than those made from conventional gold conjugation methods.

This application claims priority to U.S. provisional application No. 61/023,619 (filed Jan. 25, 2008), incorporated by reference in its entirety.

### BACKGROUND OF THE INVENTION

Gold acts as a marker for molecules that are otherwise invisible by eye or through other detection systems. A gold conjugate is formed by coupling a suspension of gold particles to a selected biological molecule such as protein (e.g., antibody). This gold label detection system, when incubated with a specific target, reveals the target through the visibility of the gold particles themselves. Thus, gold is an important tool for detection and quantification of biomolecules when combined with other known techniques such as blotting. More recently, gold conjugates have been incorporated into rapid test immunoassays. In these techniques, the unique red color of the accumulated gold label, when observed by lateral flow along a membrane on which an antigen is captured, or by measurement of the red color intensity in solution, can provide a sensitive method for detecting sub-nanogram quantities of proteins in solution.

The conditions under which a gold conjugate is made affect its performance. The present invention provides a method of conjugating gold to biological molecules. The gold conjugates produced in accordance with the present methods have an improved signal to noise ratio over commercially available gold conjugates.

### BRIEF SUMMARY OF THE INVENTION

In one aspect, the invention provides methods of conjugating gold particles to a biomolecule by contacting the gold particles with the biomolecule in a solution to form a biomolecule-gold conjugate and curing the biomolecule-gold conjugate for a period of at least 6 hours. In some cases, the biomolecule is a protein.

In another aspect, some such methods further comprise contacting the biomolecule-gold conjugate with a target, and determining whether the biomolecule-gold conjugate binds to the target. In some cases, lateral flow assay is used for this step.

In yet another aspect, some such methods provide contacting the gold particles with the biomolecule in solutions of different pH to form different aliquots of biomolecule-gold conjugate. Other such methods provide contacting different amounts of the biomolecule with the gold particles and determining the least amount of the biomolecule that avoids precipitation or aggregation of the gold particles.

Some such methods provide that the biomolecule-gold conjugate is cured at temperatures between 25° C. and 45° C. In some cases, the curing is performed for 10-20 hours. In some other cases, the biomolecule-gold conjugate is cured at 37° C.

In another aspect, some such methods provide that the biomolecule is an antibody. Such methods comprise contacting the gold particles with the antibody at pH 5.5 to 6.5 to form an antibody-gold conjugate. In some cases, the antibody is an F18-8G11 antibody. Other such methods provide that 3.5 to 4.5 μg antibody is contacted per ml OD530 of gold particles.

In some cases the gold particles of such methods are 40 nm gold particles.

In another aspect, some such methods further comprise contacting the biomolecule-gold conjugate with a blocking agent before the curing step. In some cases, the blocking agent is bovine serum albumin. In some other cases the blocking agent is contacted with the biomolecule gold conjugate at a pH from 8.5 to 9.5.

The invention also includes a biomolecule-gold conjugate produced from the methods described above.

Also provided are methods of detecting a target by contacting the target with a gold-conjugated biomolecule, wherein the biomolecule was conjugated to gold to form the conjugate and the gold-conjugated biomolecule was cured for at least 12 hours before the contacting step; and detecting a signal from the gold-conjugated biomolecule bound to the target to indicate presence of the target. In some cases, the gold-conjugated biomolecule of such methods is a reporter biomolecule. In some such methods, the target is also contacted with an immobilized PDZ domain that binds to a different epitope of the target than the reporter biomolecule. In some cases, the target is contacted with the gold-conjugated biomolecule in a lateral flow assay.

In one aspect, the biomolecule for such methods of detecting a target is an antibody. Some such methods comprise contacting the target with a gold-conjugated antibody, wherein the antibody was conjugated to gold at pH 5.5 to 6.5. In some cases, the gold-conjugated antibody is a reporter antibody. In some such methods, the target is also contacted with an immobilized PDZ domain binding to a different epitope of the target than the reporter antibody. In some cases, the target is contacted with the gold-conjugated antibody in a lateral flow assay.

### BRIEF DESCRIPTION OF THE DRAWINGS

FIG. 1 shows the optimal concentrations for the F18-8G11 antibody for gold conjugation at pH 6, 7, 8 and 9.

FIG. 2 shows a lateral flow assay using PDZ capture and the antibody-gold conjugate detection. A PDZ domain protein, MAGI 1 protein (Membrane Associated Guanylate kinase Inverted) which binds to human papillomavirus, HPV16-E6, was used as a capture protein for the lateral flow assay. The analytes included 1 and 0 ng of HPV16-maltose binding protein [MBP]-E6. This figure shows that pH6 and pH7 gave the best signal to noise ratios compared to those of pH 8 and 9.

FIG. 3 shows that curing of the antibody-gold conjugate at 37° C. overnight increases sensitivity.

FIG. 4 shows a lateral flow assay using PDZ capture and the antibody-gold conjugate detection. A PDZ domain protein, MAGI 1 protein (Membrane Associated Guanylate kinase Inverted) which binds to human papillomavirus, HPV16-E6, was used as a capture protein for the lateral flow assay. The analytes included 2.5, 0.5, 0.1 and 0 ng of HPV16-maltose binding protein [MBP]-E6.

FIG. 5 shows that the gold-conjugated antibody is significantly more sensitive than a commercially available conjugate in all test levels of the analytes.

### DEFINITIONS

The term “antibody” is used to include intact antibodies and binding fragments thereof. Typically, fragments compete with the intact antibody from which they were derived for specific binding to an antigen fragment, and can include separate heavy chains, light chains Fab, Fab′ F(ab′)2, Fabc, and Fv. Fragments are produced by recombinant DNA techniques, or by enzymatic or chemical separation of intact immunoglobulins. The term “antibody” also includes one or more immunoglobulin chains that are chemically conjugated to, or expressed as, fusion proteins with other proteins. The term “antibody” also includes bispecific antibody. A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

An “antigen” is an entity to which an antibody specifically binds.

“PDZ protein”, used interchangeably with “PDZ-domain containing polypeptides” and “PDZ polypeptides”, means a naturally occurring or non-naturally occurring protein having a PDZ domain (supra). Representative examples of PDZ proteins include CASK, MPP1, DLG1, DLG2, PSD95, NeDLG, TIP-33, TIP-43, LDP, LIM, LIMK1, LIMK2, MPP2, AF6, GORASP1, INADL, KIAA0316, KIAA1284, MAGI1, MAST2, MINT1, NSP, NOS1, PAR3, PAR3L, PAR6 beta, PICK1, Shank 1, Shank 2, Shank 3, SITAC-18, TIP1, and ZO-1. The term “PDZ domain” refers to protein sequence (i.e., modular protein domain) of less than approximately 90 amino acids, (i.e., about 80-90, about 70-80, about 60-70 or about 50-60 amino acids), characterized by homology to the brain synaptic protein PSD-95, the Drosophila septate junction protein Discs-Large (DLG), and the epithelial tight junction protein ZO1 (ZO1). PDZ domains are also known as Discs-Large homology repeats (“DHRs”) and GLGF repeats. PDZ domains generally appear to maintain a core consensus sequence (Doyle, D. A., 1996, Cell 85: 1067-76).

“PL protein” or “PDZ Ligand protein” refers to a polypeptide that may be a naturally-occurring or non-naturally occurring peptide that binds to or forms a molecular complex with a PDZ-domain. As used herein, a “PL region” or “PL” is a peptide having a sequence from, or based on, the sequence of the C-terminus of a PL protein. Representative examples of PL have been provided previously in US 20050255460 and US 20070099199.

The term “human papillomavirus” or “HPV” refers to a diverse group of DNA-based viruses that are one of the most common causes of sexually transmitted disease in the world. Cervical cancer is identified to be caused by HPV. The more than 100 different isolates of HPV have been broadly subdivided into high-risk and low-risk subtypes based on their association with cervical carcinomas or with benign cervical lesions or dysplasias. The strain HPV16 is a high-risk type of HPV, and is often accompanied by infections such as lichen sclerosis and other strains of human papilloma virus. E6 is a protein produced by the HPV16 virus. See e.g., U.S. 20030143679, 20030105285 and 20050142541; and U.S. Pat. Nos. 6,610,511, 6,492,143 6,410,249, 6,322,794, 6,344,314, 5,415,995, 5,753,233, 5,876,723, 5,648,459, 6,391,539, 5,665,535 and 4,777,239.

The term “specific binding” refers to binding between two molecules, for example, a ligand and a receptor, characterized by the ability of a molecule (ligand) to associate with another specific molecule (receptor) even in the presence of many other diverse molecules, i.e., to show preferential binding of one molecule for another in a heterogeneous mixture of molecules. Specific binding of a ligand to a receptor is also evidenced by reduced binding of a detectably labeled ligand to the receptor in the presence of excess unlabeled ligand (i.e., a binding competition assay). Specific binding between a binding agent, e.g., an antibody or a PDZ domain refers to the ability of a capture- or detection-agent to preferentially bind to a particular analyte that is present in a mixture of different analytes. Specific binding also means a dissociation constant (KD) that is less than about 10⁻⁶ M; preferably, less than about 10⁻⁷ M; and, most preferably, less than about 10⁻⁸ M. In some methods, a specific binding interaction is capable of discriminating between proteins having or lacking a PL with a discriminatory capacity greater than about 10- to about 100-fold; and, preferably greater than about 1,000- to about 10,000-fold.

The term “capture agent” or “capture reagent” refers to an agent that binds an analyte through an interaction that is sufficient to permit the agent to bind and concentrate the analyte from a homogeneous mixture of different analytes. The binding interaction is typically mediated by an affinity region of the capture agent. Typical capture agents include any protein, e.g., a PDZ protein, however antibodies may be employed. Capture agents usually “specifically bind” one or more analytes, e.g., an HPV16-E6 protein.

The term “analyte” refers to a known or unknown component of a sample, which specifically binds to a capture agent if the analyte and the capture agent are members of a specific binding pair. In general, analytes are biopolymers, i.e., an oligomer or polymer such as an oligonucleotide, a peptide, a polypeptide, an antibody, or the like. In this case, an “analyte” is referenced as a moiety in a mobile phase (typically fluid), to be detected by a “capture agent” which, in some embodiments, is bound to a substrate, or in other embodiments, is in solution. However, either of the “analyte” or “capture agent” may be the one which is to be evaluated by the other (thus, either one could be an unknown mixture of analytes, e.g., polypeptides, to be evaluated by binding with the other).

“Capture agent/analyte complex” is a complex that results from the specific binding of a capture agent, with an analyte, e.g. human papillomavirus HPV16-maltose binding protein [MBP]-E6. A capture agent and an analyte specifically bind, i.e., the one to the other, under conditions suitable for specific binding, wherein such physicochemical conditions are conveniently expressed e.g. in terms of salt concentration, pH, detergent concentration, protein concentration, temperature and time. The subject conditions are suitable to allow binding to occur e.g. in a solution; or alternatively, where one of the binding members is immobilized on a solid phase. Representative conditions so-suitable are described in e.g., Harlow and Lane, “Antibodies: A Laboratory Manual”, Cold Spring Harbor Laboratory, Cold Spring Harbor, N.Y. (1989). Suitable conditions preferably result in binding interactions having dissociation constants (KD) that are less than about 10⁻⁶ M; preferably, less than about 10⁻⁷ M; and, most preferably less than about 10⁻⁸ M.

“Solid phase” means a surface to which one or more reactants may be attached electrostatically, hydrophobically, or covalently. Representative solid phases include e.g.: nylon 6; nylon 66; polystyrene; latex beads; magnetic beads; glass beads; polyethylene; polypropylene; polybutylene; butadiene-styrene copolymers; silastic rubber; polyesters; polyamides; cellulose and derivatives; acrylates; methacrylates; polyvinyl; vinyl chloride; polyvinyl chloride; polyvinyl fluoride; copolymers of polystyrene; silica gel; silica wafers glass; agarose; dextrans; liposomes; insoluble protein metals; and, nitrocellulose. Representative solid phases include those formed as beads, tubes, strips, disks, filter papers, plates and the like. Filters may serve to capture analyte e.g. as a filtrate, or act by entrapment, or act by covalently binding. A solid phase capture reagent for distribution to a user may consist of a solid phase coated with a “capture reagent”, and packaged (e.g., under a nitrogen atmosphere) to preserve and/or maximize binding of the capture reagent to an analyte in a biological sample.

Biological samples include tissue fluids, tissue sections, biological materials carried in the air or in water and collected there from e.g. by filtration, centrifugation and the like, e.g., for assessing bioterror threats and the like. Alternative biological samples can be taken from fetus or egg, egg yolk, and amniotic fluids. Representative biological fluids include urine, blood, plasma, serum, cerebrospinal fluid, semen, lung lavage fluid, feces, sputum, mucus, water carrying biological materials and the like. Alternatively, biological samples include nasopharyngeal or oropharyngeal swabs, nasal lavage fluid, tissue from trachea, lungs, air sacs, intestine, spleen, kidney, brain, liver and heart, sputum, mucus, water carrying biological materials, cloacal swabs, sputum, nasal and oral mucus, and the like. Representative biological samples also include foodstuffs, e.g., samples of meats, processed foods, poultry, swine and the like. Biological samples also include contaminated solutions (e.g., food processing solutions and the like), swab samples from out-patient sites, hospitals, clinics, food preparation facilities (e.g., restaurants, slaughter houses, cold storage facilities, supermarket packaging and the like). Biological samples may also include in situ tissues and bodily fluids (i.e., samples not collected for testing). The biological sample may be derived from any tissue, organ or group of cells of the subject. In some embodiments a scrape, biopsy, or lavage is obtained from a subject. Biological samples may include bodily fluids such as blood, urine, sputum, and oral fluid. Optionally, the biological sample may be suspended in an isotonic solution containing antibiotics such as penicillin, streptomycin, gentamycin, and mycostatin.

“Isolated” or “purified” generally refers to isolation of a substance (compound, polynucleotide, protein, polypeptide, polypeptide composition) such that the substance comprises a significant percent (e.g., greater than 2%, greater than 5%, greater than 10%, greater than 20%, greater than 50%, or more, usually up to about 90%-100%) of the sample in which it resides. In certain embodiments, a substantially purified component comprises at least 50%, 80%-85%, or 90-95% of the sample. Techniques for purifying polynucleotides and polypeptides of interest are well-known in the art and include, for example, ion-exchange chromatography, affinity chromatography and sedimentation according to density. Generally, a substance is purified when it exists in a sample in an amount, relative to other components of the sample that is not found naturally.

“Subject”, is used herein to refer to a man and domesticated animals, e.g. mammals, fishes, birds, reptiles, amphibians and the like.

The term “epitope” or “antigenic determinant” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed. (1996). Antibodies that recognize the same epitope can be identified in a simple immunoassay showing the ability of one antibody to block the binding of another antibody to a target antigen. T-cells recognize continuous epitopes of about nine amino acids for CD8 cells or about 13-15 amino acids for CD4 cells. T cells that recognize the epitope can be identified by in vitro assays that measure antigen-dependent proliferation, as determined by ³H-thymidine incorporation by primed T cells in response to an epitope (Burke et al., J. Inf. Dis. 170, 1110-19 (1994)), by antigen-dependent killing (cytotoxic T lymphocyte assay, Tigges et al., J. Immunol. 156, 3901-3910) or by cytokine secretion. The epitope of a monoclonal antibody (mAb) is the region of its antigen to which the mAb binds.

The terms “sandwich”, “sandwich ELISA”, “sandwich diagnostic” and “capture ELISA” all refer to the concept of detecting a biological polypeptide with two different test agents. For example, a PDZ protein can be directly or indirectly attached to a solid support. Test sample can be passed over the surface and the PDZ protein can bind its cognate protein(s). A gold-conjugated antibody or alternative detection reagent can then be used to determine whether the specific protein has bound the PDZ protein.

Detecting “presence” or “absence” of an analyte includes quantitative assays in which only presence or absence of analyte is detected and quantitative assays in which presence of analyte is detected as well as an amount of analyte present.

A “biomolecule” is a molecule having a type of structure found in living organism (e.g., proteins, particularly antibodies, carbohydrates, lipids, and nucleic acid). A molecule can be considered a biomolecule irrespective of whether it occurs in nature. For example, monoclonal antibodies, chimeric, and humanized antibodies are biomolecules because they are proteins. Likewise, a cDNA is biomolecule because it is a nucleic acid.

The term “marker” or “biological marker” refers to a measurable or detectable entity in a biological sample. Examples or markers include nucleic acids, proteins, or chemicals that are present in biological samples. One example of a marker is the presence of viral or pathogen proteins or nucleic acids in a biological sample from a human source.

### DETAILED DESCRIPTION OF THE INVENTION

### A. General

The invention provides improved methods of conjugating gold to biological molecules and conjugates resulting from the same. The results presented in the Examples show that gold conjugates produced in accordance with these methods have an improved signal to noise ratio over commercially available gold conjugates. Although practice of the invention is not dependent on an understanding of mechanism, it is believed that the increased sensitivity resides at least in part from a step of incubating conjugates at above room temperature after formation and before use (“curing”). The methods are particularly suitable for forming antibody-gold conjugates. Conjugates formed in accordance with the methods of the invention can be used in the same applications as conventional gold conjugates.

### B. Colloidal Gold and Gold Particles

Colloidal gold is a suspension of gold particles in a fluid. The gold particles can come in a variety of shapes such as spheres, rods and cubes, but preferably, spheres. Gold particles usually range in size from 1-250 nm. For particles less than 100 nm, the liquid is usually red. For larger particles, the color is of a dirty yellowish color. In principle, smaller gold particles produce a higher labeling intensity on the specimen because of the reduced stearic hinderance to target detection. However, different particle sizes are appropriate for different applications. For example, 1-5 nm gold particles are recommended for intracellular staining because they are able to penetrate the cell membranes more easily. 1-5 nm gold particles are also recommended for high resolution electron microscopy (EM) because the small particle size allows for more precise localization of the antigen. 5-10 nm particles are recommended for cell surface staining and for light microscopy because the larger size makes the stain more visible. 20-50 nm particles are recommended for some histochemical applications and for blotting.

Gold particles can be obtained commercially from many sources such as British BioCell International, Ltd (BBI) (Cardiff, United Kingdom); Millenia Diagnostic, Inc. (San Diego, Calif.), EBSTed Pella, Inc. (Redding, Calif., USA); SPI Supplies and Structure Probe, Inc. (West Chester, Pa., USA); and Sigma-Aldrich Company (Saint Louis, Mo., USA).

There are several advantages in choosing gold particles as markers for identification of target molecules. Gold is stable, nontoxic, safe and easy to use. Gold gives a permanent label unlike fluorescent labels and enzyme-based color labels which fade over time and light exposure. For the staining of proteins immobilized onto membranes, gold has unsurpassed detection sensitivity and resolution. Gold is inexpensive in its application and its high sensitivity allows valuable primary antibodies to be diluted significantly further than other systems.

### C. Biomolecules

Gold can be conjugated to a wide variety of molecules including proteins (e.g., antibodies, enzymes), carbohydrates, polysaccharides, nucleic acids and polymers. The conditions under which a gold conjugate is made significantly affect its performance and stability.

The conjugation of proteins to gold particles can be effected by any or all of at least three physical phenomena: (1) charge attraction of the negative gold particle to positively charged protein; (2) hydrophobic absorption of the protein to the gold particle surface; and (3) dative binding between the gold conducting electrons and sulfur atoms which may exist within amino acids of the protein (e.g., cysteine or methionine).

Several features are useful to provide high quality gold conjugates. The biomolecule to be conjugated to gold should be of high quality. If the biomolecule is a protein, it is desirable that the protein purified; preferably, affinity purified. Also, the protein preferably has a strong affinity for the specific target molecule to be detected and has high avidity to withstand severe incubation and washing conditions. If an antibody is used, antigenic cross reactivity is preferably minimized. Gold particles used for conjugation preferably have the lowest available coefficient of variation (CV) to ensure size uniformity. High optical density, purity and long shelf life are also preferable.

Sensitivity is important in immunoassays for detection of low levels of antigens. The gold particle on the antibody-gold conjugates should minimally affect the activity of the antibody to which it is conjugated but be strong enough to be absorbed to the surface to remain stable for years. For long-term storage, an antibody-gold conjugate can be lyophilized for years without loss of antibody activity.

### D. Methods of Conjugating Gold Particles to Biomolecules

The basic steps involve combining a biomolecule with gold particles in solution by a method, such as rocking, shaking or vortexing the container or stirring the solution. The conjugation process can last for a period of time, such as for example, ranging from a second to 30 minutes and can be performed at an appropriate temperature conditions, for example ranging from 4° C. to 45° C. Preferably, the conjugation process is performed at room temperature. The biomolecule and gold particles combine by non-covalent interactions to form a biomolecule-gold conjugate. Binding sites on the gold particles that are not bound to the biomolecule of interest are then blocked, usually with a readily available protein unrelated to the biomolecule of interest (e.g., bovine serum albumin). The biomolecule-gold conjugate is purified from excess biomolecule and gold by methods such as centrifugation, fractionation or gel filtration.

The present inventors have discovered a variation of the basic procedure resulting in an improved signal to noise ratio of the resulting conjugates. The improvement involves incubating conjugates at above room temperature after formation but before use. The conjugates are typically subjected to elevated temperatures after separation from unused reagents used in their formation. Typically, such conjugates contain a biomolecule, gold particles and a blocking agent. Although practice of the present invention is not dependent on an understanding of mechanism, it is believed that a period of exposure to an elevated temperature strengthens noncovalent bonding between the gold particles and the biomolecule of interest and/or between the gold particles and the blocker, and that such strengthened bonding leads to an improved signal to noise ratio. After exposure to an elevated temperature, the conjugates can be used immediately, or stored in the cold or lyophilized until use. The exposure to elevated temperature is in contrast to previous methods in which conjugates are used immediately or stored in the cold or below zero temperatures before use.

The process of incubating conjugates at elevated temperatures is referred to as “curing.” Conjugates are exposed to elevated temperature for at least 6, 12, or 24 hours. 6-24 hours, or more preferably 10-20 hours, is usually a sufficient period. In an embodiment, curing can be performed for a short duration, such as 1 minute, 2 minutes, 5 minutes, 20 or 30 minutes, or about one hour. Optionally the curing is at elevated temperatures. Elevated temperature means a temperature above room temperature (i.e., above 20° C. and preferably 21-50° C. or 30-45° C.). Curing can be performed for example by an overnight incubation in a 37° C. incubator. On occasion, the curing can be done at non-elevated temperatures, such as at ambient or room temperatures, e.g., about 15° C. or 2 at about 20° C.). In an amendment, the curing step is not longer than about 5 hours. For example, the curing is not longer than about 1 or 2 hours, on occasion not longer than about 30 minutes, for example not longer than about 5 minutes or 10 minutes. Optionally the curing step is in the range of about 1 minutes-5 hours, for example in the range of about 30 minutes to 2 hours, or in the range of about 10 minutes to 1 hour, or about 1-30 minutes, for example 1 to 10 minutes, sometimes 1 to 5 minutes.

The increase in signal to noise ratio effected by curing is particularly useful when the biomolecule is an antibody. Preferred antibodies for use in the invention include the F18-8G11 antibody that specifically binds to human papillomavirus HPV16-E6 as well as antibodies against the influenza NS1 protein used in diagnosis of influenza (see WO2007018843 and US2005142541).

The curing step is preferably performed in combination with the optimization of pH for formation of conjugates. The pH can affect the nature and extent of noncovalent binding between the biomolecule and the gold particles, and between the gold particles and the blocker. For any given biomolecule, the optimal pH once determined, need not be re-determined. However, the optimum pH can vary for different biomolecules. A useful starting point for some biomolecules is to use a pH at or close to the pI of the biomolecule. For example, some antibodies have a pI between pH 5 and 6. However, it is recommended that optimal pH be determined empirically by forming conjugates at a range of pH's usually between 3 and 11 in increments of 1 or 0.5 units. Conjugates formed at the different pH's are cured and tested in a standard assay to identify which conjugate gives the highest signal to noise ratio. The pH at which that conjugate was formed is identified as being the optimal pH to use in conjugating additional batches of the biomolecule.

After the biomolecule is conjugated to the gold particles, a solution of suitable blocker/stabilizer such as inert proteins e.g., bovine serum albumin (BSA), blood substitute mixtures, or polyethylene glycols, is added. This stabilization is for reducing aggregation of the excess free gold particles and the biomolecule-conjugated gold particles and for saturating remaining free surfaces of the biomolecule that are accessible to the gold particles. (Beesley, supra; Behnke, Eur. J. Cell. Bio. 41 (1986), 326-338). The blocking step is usually performed at the same pH as the conjugation step, but can be performed at a different pH. If performed at a different pH, the pH can be the pI of the blocker, or can be optimized empirically. The pH of the blocking solution may vary such as from pH 6 to pH 10. Generally, the blocking step can be performed at room temperature and the subsequent centrifugation step and resuspension can be done at a colder temperature.

The curing step is also preferably performed in combination with optimization of the ratio between the biomolecule and the gold particles. The optimal ratio of biomolecule to gold particle is the lowest ratio at which the gold particles remain in colloidal form in solution in the presence of 1% salt. Too few biomolecules result in unsuccessful conjugation. Too many biomolecules result in unlabelled biomolecules competing with labeled biomolecules for binding to a target, reducing the signal to noise ratio. The optimization of ratio is preferably also performed in combination with an optimization of pH. The ratio and pH can be optimized at the same time or sequentially. Furthermore, the optimal amount of the biomolecules and gold required for conjugation also varies based on the pH of the conjugation. For example, the ratio and pH can be optimized simultaneously by titrating the minimum amount of antibody needed to keep the same amount of gold particles in solution in different solutions at a range of pH's.

A preliminary titration of different amounts of antibody and gold (e.g., 1 μg to 10 μg of antibody per OD per mL of gold) under a given pH condition is preferable for successful conjugation.

The steps of curing, pH optimization and optimizing the ratio of biomolecule to gold particles are preferably performed in combination with one another, but can also be introduced independently into the basics procedure for conjugate formation described above.

After formation of conjugates, the quantity and quality of the biomolecule-gold conjugate, can be determined by electron microscopy or by spectrophotometrical means. Commercially available gold particles are measured in optical density units of 520 nm (see BBI catalog and manual); however, the optimal peak absorbance wavelength of the biomolecule-gold conjugate can vary somewhat from 520 nm. Thus, a preliminary measurement of the biomolecule-gold conjugates at different absorbance wavelengths such as from 450 nm to 650 nm can be conducted.

For antibody-gold conjugates, the quantity of the antibody-gold conjugate can also be measured by immunoassays such as immunoblotting against the specific antigen on biological materials (e.g., tissue sections or cells) or against specific antigen immobilized on non-biological materials (e.g., nitrocellulose, plastic). The biomolecule-gold conjugates can then adjusted to the appropriate concentrations based on the specific application of the antibody-gold conjugate.

Quality control of biomolecule-gold conjugates is useful in comparing conjugates of the same biomolecule formed under different conditions (e.g., different pH) or assessing one batch of a conjugate for consistency with another or a standard. A standard binding assay, such as an ELISA or lateral flow assay is useful for this purpose. The signal to noise ratio can be assessed by measuring the difference in signal intensities between titrated biomolecule-gold conjugates and a negative control on a CAMAG machine.

After formation (including curing if performed) the biomolecule-gold conjugate is usually stable at 4° C. for several months. For long term storage, the conjugate can be aliquoted into smaller volumes with agents such as glycerol or polymers which have good freezing characteristics added. The conjugates can then be lyophilized.

### E. Applications

The invention provides diagnostic capture and detect reagents useful in assay methods for identifying biological molecule targets in a variety of different types of biological samples. The methods of the invention are also useful for a variety of other diagnostic analyses, therapeutic methods and experimental research. The formats to visualize the gold conjugates include electron microscopy (EM) such as transmission electron microscopy (TEM) and scanning electron microscopy (SEM), light microscopy (LM) and the naked eye. See J. Roth, The Colloidal Gold Marker System for Light and Electron Microscopic Cytochemistry in: Immunocytochemistry 2 (1983) pp. 218-284. Gold-antibody conjugates can be used in immunoassays such as immunohistochemistry, immunocytochemistry and immunoblotting. Such immunoassays are sometimes performed in combination with silver enhancement to amplify the signal for visualization under light microscopy or with the naked eye. Gold-antibody conjugates can also be used in formats such as immunoprecipitation, Western blotting, ELISA, radioimmunoassay, competitive and immunometric assays and lateral flow assays. See Harlow & Lane, Antibodies, A Laboratory Manual (CSHP NY, 1988); U.S. Pat. Nos. 3,791,932; 3,839,153; 3,850,752; 3,879,262;4,034,074, 3,791,932; 3,817,837; 3,839,153; 3,850,752; 3,850,578; 3,853,987; 3,867,517; 3,879,262; 3,901,654; 3,935,074; 3,984,533; 3,996,345; 4,034,074; 4,098,876; 4,376,110; 4,486,530; 5,914,241 and 5,965,375.

Lateral flow devices are a preferred format. Similar to a home pregnancy test, lateral flow devices work by applying fluid to a test strip that has been treated with specific biologicals. Carried by the liquid sample, phosphors labeled with corresponding biologicals flow through the strip and can be captured as they pass into specific zones. The amount of phosphor signal found on the strip is proportional to the amount of the target analyte. The lateral flow typically contains a solid support (for example nitrocellulose membrane) that contains three specific areas: a sample addition area, a capture area, and a read-out area that contains one or more zones, each zone containing one or more labels. The lateral flow can also include positive and negative controls. Thus, for example a lateral flow device can be used as follows: target proteins are separated from other proteins in a biological sample by bringing an aliquot of the biological sample into contact with one end of a test strip, and then allowing the proteins to migrate on the test strip, e.g., by capillary action such as lateral flow. Proteins, antibodies, and/or aptamers are included as capture and/or detect reagents. Methods and devices for lateral flow separation, detection, and quantification are known in the art, e.g., U.S. Pat. Nos. 6,942,981, 5,569,608; 6,297,020; and 6,403,383 incorporated herein by reference in their entirety.

One form of a lateral assay is a PDZ capture assay. In such an assay, a PDZ protein or one antibody or population of antibodies is immobilized to a solid phase as a capture agent, and another antibody or population of antibodies or a PDZ protein in solution is as detection agent. In one non-limiting example, a test strip comprises a proximal region for loading the sample (the sample-loading region) and a distal test region containing a PDZ protein capture reagent and buffer reagents and additives suitable for establishing binding interactions between the PDZ protein and any PL protein in the migrating biological sample. The selection of PDZ capture reagent and antibody detection reagent depends on the target. Typically, the detection agent is labeled, such as with gold. If an antibody population is used, the population typically contains antibodies binding to different epitope specificities within the target antigen. Accordingly, the same population can be used for both capture agent and detector agent. If monoclonal antibodies are used as detection and detection agents, first and second monoclonal antibodies having different binding specificities are used for the solid and solution phase. Capture and detection agents can be contacted with target antigen in either order or simultaneously. If the capture agent is contacted first, the assay is referred to as being a forward assay. Conversely, if the detection agent is contacted first, the assay is referred to as being a reverse assay. If target is contacted with both capture agent and detection agent simultaneously, the assay is referred to as a simultaneous assay. After contacting the sample with capture and detection antibodies, a sample is incubated for a period that usually varies from about 10 min to about 24 hr and is usually about 1 hr. A wash step can then be performed to remove components of the sample not specifically bound to the detection agent. When capture and detection agents are bound in separate steps, a wash can be performed after either or both binding steps. After washing, binding is quantified, typically by detecting label linked to the solid phase through binding of labeled solution antibody. Usually for a given pair of capture and detection agents and given reaction conditions, a calibration curve is prepared from samples containing known concentrations of target antigen. Concentrations of antigen in samples being tested are then read by interpolation from the calibration curve. Analyte can be measured either from the amount of labeled solution antibody bound at equilibrium or by kinetic measurements of bound labeled solution antibody at a series of time points before equilibrium is reached. The slope of such a curve is a measure of the concentration of target in a sample.

Competitive assays can also be used. In some methods, target antigen in a sample competes with exogenously supplied labeled target antigen for binding to an antibody or PDZ detection reagent. The amount of labeled target antigen bound to the detection reagent is inversely proportional to the amount of target antigen in the sample. The detection reagent can be immobilized to facilitate separation of the bound complex from the sample prior to detection (heterogeneous assays) or separation may be unnecessary as practiced in homogeneous assay formats. The detection reagent is labeled with gold and its binding sites compete for binding to the target antigen in the sample and an exogenously supplied form of the target antigen that can be, for example, the target antigen immobilized on a solid phase. Gold labeled detection reagent can also be used to detect antibodies in a sample that bind to the same target antigen as the labeled detection reagent in yet another competitive format. In each of the above formats, the detection reagent is present in limiting amounts roughly at the same concentration as the target that is being assayed.

A preferred format uses the PDZ capture assay for detection of human papillomavirus (HPV) or influenza. For example, a sample suspected of containing human papillomavirus or influenza virus is added to a lateral flow device, the sample is allowed to move by diffusion and a line or colored zone indicates the presence of the human papillomavirus or the influenza virus. The lateral flow typically contains a solid support (for example nitrocellulose membrane) that contains three specific areas: a sample addition area, a capture area containing one or more antibodies to human papillomavirus HPV16-E6 such as the F18-8G11 antibody, and a read-out area that contains one or more zones, each zone containing one or more labels. The lateral flow can also include positive and negative controls. Thus, for example a lateral flow device can be used as follows: human papillomavirus HPV16-E6 proteins are separated from other viral and cellular proteins in a biological sample by bringing an aliquot of the biological sample into contact with one end of a test strip, and then allowing the proteins to migrate on the test strip, e.g., by capillary action such as lateral flow. A preferred format for detection of HPV16-E6 employs a PDZ protein such as MAGI 1, as the capture reagent, which binds to a PL in the target, HPV16-E6 protein and an antibody such as the gold-conjugated F18-G11 antibody which binds to a different epitope on MAGI1 as a detection antibody. The same or different antibody can be used with different PDZ proteins in the same assay. See e.g., US 20050142541, 20070014803, 20070099199 and 20070161078.

Gold-conjugated biomolecules for use in the above methods are detectable by spectroscopic, photochemical, biochemical, immunochemical, electrical, optical, chemical, or other means.

The level of the human papillomavirus HPV16-E6 protein in a sample can be quantified and/or compared to controls. Suitable negative control samples are e.g. obtained from individuals known to be healthy, e.g., individuals known not to have a human papillomavirus viral infection. Specificity controls may be collected from individuals having known human papillomavirus HPV16 infection, or individuals infected with viruses other than human papillomavirus HPV16. Control samples can be from individuals genetically related to the subject being tested, but can also be from genetically unrelated individuals. A suitable negative control sample can also be a sample collected from an individual at an earlier stage of infection, i.e., a time point earlier than the time point at which the test sample is taken. Recombinant human papillomavirus HPV16-[MBP]-E6 can be used as a positive control.

The sensitivity level of the detection reagent can also be measured with the lateral flow format by varying the amount of target analytes in the assay. For example, target analyte human papillomavirus HPV16-[MBP]-E6 at 2.5, 0.5, and 0.1 ng levels are used to determine the sensitivity of the gold-conjugated F18-8G11 antibody.

All publications, and patent filings cited in this specification are herein incorporated by reference as if each individual publication or patent were specifically and individually indicated to be incorporated by reference. Unless otherwise apparent from the context, any feature, step or embodiment can be used in combination with any other feature, step or embodiment.

### EXAMPLES

### Example 1

### Optimization of the PH and the Amount of the Antibody for Gold Conjugation of the F18-8G11 Antibody

stock solutions of an antibody termed F18-8G11 were prepared by diluting the F18-8G11 antibody in 4 antibody buffers. For each pH optimization step, 90 μL of the F18-8G11 antibody was combined with 10 μL of the antibody buffer (1M MES pH6, 1M PIPES pH7, 1M Tricine pH8, or 1M TAPS pH9), to result in a final concentration of 1.341 mg/mL of the F18-8G11 antibody in 100 mM of antibody buffer (“F18-8G11 antibody working solution”).

4 stock solutions of colloidal gold particles were prepared by diluting the 40 nm gold particles purchased from BBI (OD=1.0) in 4 gold buffers. For each pH optimization step, 20 mL of the gold particle stock solution was combined with 80 μL of the gold buffer (1M MES pH6, 1M PIPES pH7, 1M Tricine pH8, or 1M TAPS pH9) to result in a final concentration of approximately OD=1.0 in 4 mM of gold buffer (“buffered gold particle working solution”).

The optimal concentration of the F18-8G11 antibody was determined as the “minimal” antibody loading required to stabilize an antibody-gold conjugate and prevent it from precipitating or aggregating in the presence of 1% salt. The result was measured spectophotometrically at 580 nm (OD₅₈₀). The detailed procedure is as follows. 4 optimization buffers which are 1M MES pH6, 1M PIPES pH7, 1M Tricine pH8 and 1M TAPS pH9 were diluted to a final concentration of 4 mM by combining 4 μL of the 1M buffer with 996 μl of ultra-pure water. The diluted optimization buffers were aliquotted into 11 eppendorf tubes. 0 μg to 10 μg of F18-8G11 antibody was added to each eppendorf tube at a volume between 0 μL and 7.46 μL of F18-8G11 antibody working solution. 1 mL of the buffered gold particle working solution was added to each tube. The mixture was vortexed and the eppendorf tubes was mixed on a rocking machine for 5 minutes. 100 μL of H₂O was added to the eppendorf tube containing 0 μg of F18-8G11 antibody and 100 μL of 10% NaCl was added to the other tubes. The mixture was vortexed and the tubes were rocked for 5 minutes. The absorbance of OD₅₈₀ was determined for each tube. The titration was fine tuned as necessary.

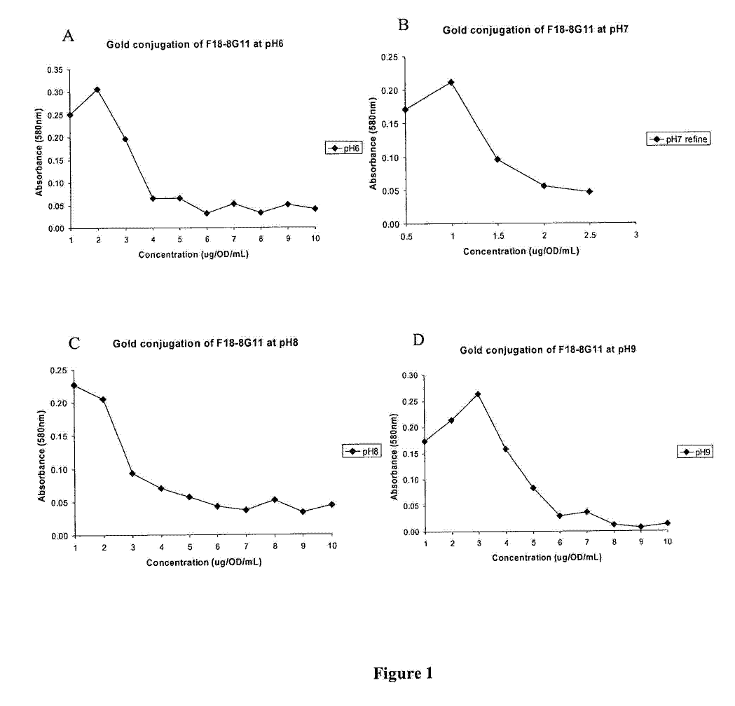

The results showed that the optimal (minimal) amount of the F18-8G11 antibody at pH 6 was 4 μg/OD/mL (see FIG. 1A). The optimal (minimal) amount at pH 7 was 2 μg/OD/mL (see FIG. 1B); the optimal (minimal) amount at pH 8 was 7 μg/OD/mL (see FIG. 1C); the optimal (minimal) amount at pH 9 was 6 μg/OD/mL (see FIG. 1D).

The units μg/OD/mL indicate the mass of antibody that was used to load an OD=1 of the gold solution per ml of the OD=1 solution. For example, if the loading was 4 μg/OD/mL, then for 1 ml of an OD=10 solution, 40 μg of antibody was used for the loading.

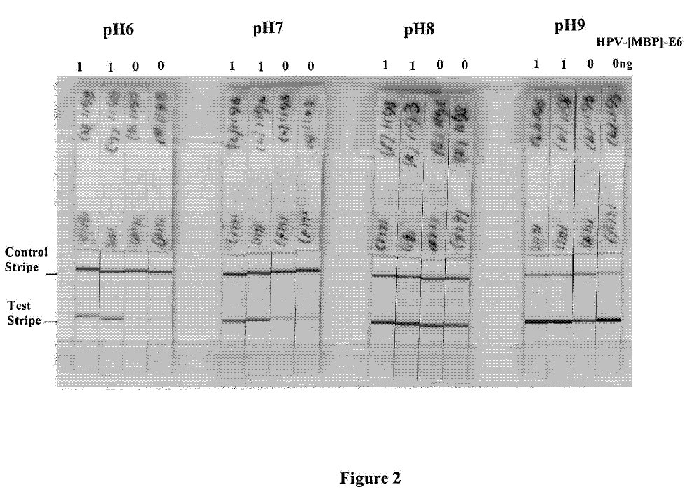

The optimal F18-8G11 antibody-gold conjugates at each of the pH conditions was tested by PDZ capture lateral flow assay, and pH6 and pH7 gave the best signal to noise ratios (see FIG. 2).

### Example 2

### The Curing Step Increases Sensitivity

The two conjugates described in Example 1 that gave the best signal to noise ratio were repeated, with or without overnight curing at 37° C. and re-tested by PDZ capture lateral flow assay. The detail of the experiment is as follows. Chimeric MAGI 1 at 3 mg/mL in 3% PIPES and 1% maltitol was deposited on the HF120 nitrocellulose membrane. For detection, 6 μL of the F18-8G11 antibody-gold conjugate were used. The analytes included 0.5 and 0 ng of HPV16-[MBP]-E6. The buffer was 100 μL of Buffer 415 (for pH6) which consisted of 5×TE0 mM Tris pH 8, 2% BSA, 2% Triton X-100, 250 mM NaCl and 40 mM EDTA; or Buffer 456 (for pH7) which consisted of 100 mM TAPS pH9, 2% BSA, 1% Triton X-100, 300 mM NaCl, 0.2% PVA-10 and 0.05% PVP-10.

The test strip was developed by fully wicking the strip in a 96-well plate for about 65 minutes in the respective Buffers containing 0.5 or 0 ng of the analytes and 6 μL the F18-8G11 antibody-gold particle conjugate. The strip was photographed with a Nikon D80 camera and signal strength was quantified using a CAMAG machine. The photograph was auto-contrasted using the Picasa software, which is a computer application for organizing and editing digital photos. (Google, Mountain View, Calif.).

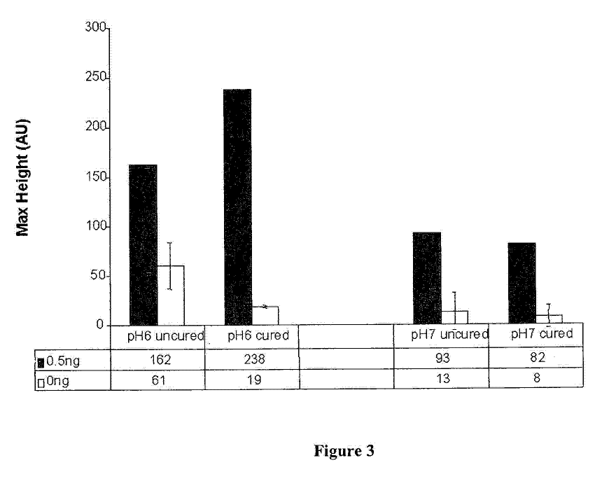

FIG. 3 shows that for both pH6 and pH7, the F18-8G11 antibody-gold conjugate that have been cured overnight at 37° C. yielded better signal to noise in the lateral flow test assay compared with the conjugates that were not cured. Other experiments also show that before curing the F18-8G11 antibody-gold conjugate at 37° C. overnight, the antibody-gold conjugate yielded a poor signal to noise in the lateral flow test assay, with high background and sometimes uneven control and tests lines. However, after curing, the antibody-gold conjugate yielded a good signal to noise in the lateral flow test with good sensitivity, and good control and test line quality.

### Example 3

### Gold Conjugation of the F18-8G11 Antibody

The material for the gold conjugation of the anti-HPV16-E6 monoclonal antibody F18-8G11 included the following: purified antibody F18-8G11; 40 nm gold particles from British BioCell International, Ltd (BBI; Cardiff, United Kingdom); 1M 2-[N-Morpholino]ethanesulfonic acid buffer (MES buffer), pH6; 10% bovine serum albumin (BSA), pH9.0 (“blocking solution”); and a resuspension solution which contains 20 mM Tris, 1% BSA and 150 mM NaCl. Purified water from VWR International (West Chester, Pa.) was used to make the buffers and solutions described above, and throughout the procedure below.

The procedure for the gold conjugation of F18-8G11 antibody was as follows. The optimal loading concentration for F18-8G11 was determined based on the experiments described in Example 1. Thus, for this experiment, the optimal loading concentration for the F18-8G11 antibody was 4 μg/OD/mL in MES pH6 buffer. The F18-8G11 antibody buffer and the gold particles buffer were prepared immediately prior to the conjugation. The F18-8G11 antibody was diluted to 0.1 mg/mL with 2 mM MES pH6 buffer. To illustrate this dilution step in greater detail, 40.1 μL of the F18-8G11 antibody (at 4.99 mg/mL) was mixed with 4 μL of 1M MES pH6 buffer and 1.958 mL of H₂O. Gold particles were brought to room temperature before adjusting to 2 mM with the 1M MES pH6 buffer. To illustrate the dilution step of the gold particles in greater detail, 30 mL of 40 nm gold particles were mixed with 60 μL of 1M MES pH6 buffer.

The F18-8G11 antibody and the gold particles were at room temperature when the conjugation occurred. The F18-8G11 antibody and the gold particles were conjugated by slowly adding an optimal amount of the F18-8G11 antibody buffer to the optimal amount of gold particles buffer while shaking the container. As an example, 1.2 mL of the F18-8G11 antibody at pH6 was mixed with 30 mL of gold particles at pH6 to obtain 4 μg/OD/mL. The conjugation mixture was mixed on a rocking machine at room temperature for 10 minutes. Approximately 10% by volume of the blocking solution was quickly added to the conjugation mixture while shaking the container. To illustrate in greater detail, 3 mL of the blocking solution was added to the F18-8G11 antibody-gold particle conjugate. The conjugation mixture was rocked with the blocking solution for 10 minutes and everything was aliquotted into eppendorf tubes. For example, 30.4 mL of the entire mixture was aliquoted into 16 eppendorf tubes (1.9 mL per tube) and the remaining 3.0 mL into 2 eppendorf tubes (1.5 mL per tube).

The wavelength for optimal absorbance was determined by diluting the F18-8G11 antibody-gold particle conjugate ten-fold by mixing 10 μL of the F18-8G11 antibody-gold particle conjugate with 90 μL of the resuspension solution. The peak absorbance wavelength was determined to be 530±2 nm.

The F18-8G11 antibody-gold particle conjugate was centrifuged at 4° C. at 7,000 rpm for 30 minutes in individual eppendorf tubes. The conjugate can also be centrifuged at a higher speed for less time. The supernatant was removed and the pellets were resuspended in a total volume of 0.5 mL of the resuspension solution by pooling the liquid along the way. The resuspension solution was kept at a low temperature. All the eppendorf tubes were rinsed with an additional 0.5 mL of the resuspension solution and the resulting liquid was added to the previous 0.5 mL liquid which contained the F18-8G11 antibody-gold particle conjugate.

The F18-8G11 antibody-gold particle conjugate was adjusted to OD₅₃₀ of 10.0. To illustrate in greater detail, the F18-8G11 antibody-gold particle conjugate was diluted twenty-fold by adding 5 μL of the F18-8G11 antibody-gold particle conjugate to 95 μL of the resuspension solution. The absorbance wavelength of the diluted F18-8G11 antibody-gold particle conjugate was measured at 530 nm. The average reading of OD₅₃₀ was determined to be 0.8421. Thus, 2 mL of the F18-8G11 antibody-gold particle conjugate was combined with 1.368 mL of the resuspension solution to obtain 3.4 mL of OD₅₃₀ of 10.0.

The diluted F18-8G11 antibody-gold particle conjugate was cured overnight at 37° C. Quality control experiments using the lateral flow assay described in Example 3 were conducted after the conjugation step.

### Example 4

### Comparison of the BBI Antibody-Gold Conjugate with the Arbor Vita Corporation (AVC) Antibody-Gold Conjugate Using the Lateral Flow Assay

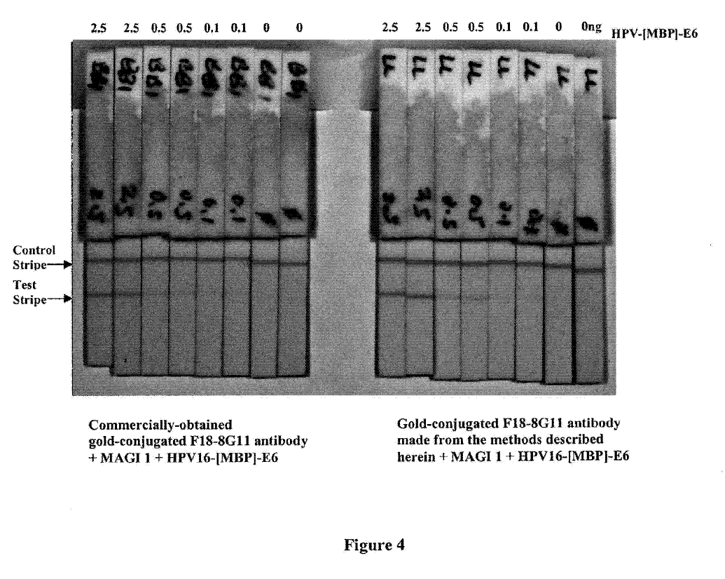

FIG. 4 shows a lateral flow assay using PDZ capture followed by the antibody-gold conjugate detection. A PDZ domain protein, Magi-1 protein (Membrane Associated Guanylate kinase Inverted) which binds to HPV16-E6, was used as a capture protein for the lateral flow assay. Chimeric MAGI 1 at 7 mg/mL in 3% PIPES and 1% maltitol was deposited on the HF135 nitrocellulose membrane as the test line, which lay 5.7 mm away from the control line. For detection, 4 μL of the BBI F18-8G11 antibody-gold conjugate and 4 μL of the AVC F18-8G11 antibody-gold conjugate were used. The analytes included 2.5, 0.5, 0.1 and 0 ng of HPV16-[MBP]-E6. The buffer was 100 μL of Buffer 597 which consists of 20 mM Tris pH 8, 2% BSA, 2% Triton X-100, 250 mM NaCl and 40 mM EDTA.

The test strip was developed by fully wicking the strip in a 96-well plate for 40 minutes in Buffer 597 containing 2.5, 0.5, 0.1 or 0 ng of the analytes and 4 μL the F18-8G11 antibody-gold particle conjugate. The strip was photographed with a Nikon D80 camera and signal strength was quantified using a CAMAG machine. The photograph was auto-contrasted using the Picasa software, which is a computer application for organizing and editing digital photos. (Google, Mountain View, Calif.).

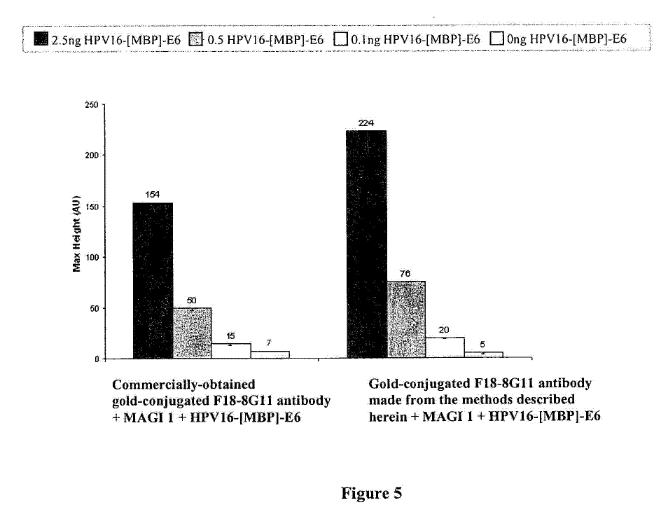

The results are shown in FIG. 5. The vertical axis of FIG. 2 indicates the maximum height reading from the CAMAG machine (in Absorbance Units or AUs). When 2.5 ng of the analyte, HPV16-[MBP]-E6 was used, the maximum height (AU) for the BBI conjugation was 154, whereas that for the AVC conjugation was 224. Experiments with 0.5 ng of HPV16-[MBP]-E6 showed that the maximum height (AU) for the BBI conjugation was 50 whereas that for the AVC conjugation was 76. Experiments using 0.1 ng of HPV16-[MBP]-E6 showed that the maximum height (AU) for the BBI conjugation was 15 whereas that for the AVC conjugation was 20. The control experiment using the 0 ng of HPV16-[MBP]-E6 showed that the maximum height (AU) for the BBI conjugation was 7 and the maximum height (AU) for the AVC conjugation was 5. Therefore, the results showed that the BBI's conjugation is significantly worse than the AVC's conjugation in all test levels of the analytes tested.

1. A method of conjugating gold particles to a biomolecule comprising:
(a) contacting the gold particles with the biomolecule in a solution to form a biomolecule-gold conjugate;
(b) curing the biomolecule-gold conjugate for a period of at least 1 minute and not longer than about 5 hours.

2. The method of claim 1, wherein the biomolecule is a protein.

3. The method of claim 1, further comprising (c) contacting the biomolecule-gold conjugate with a target, and determining whether the biomolecule-gold conjugate binds to the target.

4. The method of claim 3, wherein step (c) is a lateral flow assay.

5. The method of claim 1, wherein step (a) comprises contacting the gold particles with the biomolecule in solutions of different pH to form different aliquots of biomolecule-gold conjugate.

6. The method of claim 1, wherein step (a) comprises contacting different amounts of the biomolecule with the gold particles and determining the least amount of the biomolecule that avoids precipitation or aggregation of the gold particles.

7. The method of claim 1, wherein in step (b), the biomolecule-gold conjugate is cured at temperatures between 25° C. and 45° C.

8. The method of claim 7, wherein the curing is performed for about 5 minutes.

9. The method of claim 7, wherein the biomolecule-gold conjugate is cured at 37° C.

10. The method of claim 1, wherein the biomolecule is an antibody.

11. The method of claim 10, wherein step (a) comprises contacting the gold particles with the antibody at pH 5.5 to 6.5 to form an antibody-gold conjugate.

12. The method of claim 10, wherein the antibody is an F18-8G11 antibody.

13. The method of claim 10, wherein 3.5 to 4.5 μg antibody is contacted per ml OD530 of gold particles.

14. The method of claim 1, wherein the gold particles are 40 nm gold particles.

15. The method of claim 1, further comprising contacting the biomolecule-gold conjugate with a blocking agent before the curing step.

16. The method of claim 15, wherein the blocking agent is bovine serum albumin.

17. The method of claim 16, wherein the blocking agent is contacted with the biomolecule gold conjugate at a pH from 8.5 to 9.5.

18. A biomolecule-gold conjugate produced from the method of claim 1.

19. A method of detecting a target, comprising:
(a) contacting the target with a gold-conjugated biomolecule, wherein the biomolecule was conjugated to gold to form the conjugate and the gold-conjugated biomolecule was cured for at least 5 minutes before the contacting step;
(b) detecting a signal from the gold-conjugated biomolecule bound to the target to indicate presence of the target.

20. The method of claim 19, wherein the gold-conjugated biomolecule is a reporter biomolecule.

21. The method of claim 19 wherein the target is also contacted with an immobilized PDZ domain binding to a different epitope of the target than the reporter biomolecule.

22. The method of claim 21, wherein the target is contacted with the gold-conjugated biomolecule in a lateral flow assay.

23. The method of claim 19, wherein the biomolecule is an antibody.

24. The method of claim 23, wherein step (a) comprises contacting the target with a gold-conjugated antibody, wherein the antibody was conjugated to gold at pH 5.5 to 6.5.

25. The method of claim 24, wherein the gold-conjugated antibody is a reporter antibody.

26. The method of claim 25, wherein the target is also contacted with an immobilized PDZ domain binding to a different epitope of the target than the reporter antibody.

27. The method of claim 26, wherein the target is contacted with the gold-conjugated antibody in a lateral flow assay.
